(19) United States
(12) Patent Application Publication
(10) Pub. No.: US 2025/0288339 A1
(43) Pub. Date: Sep. 18, 2025

(54) BONE CEMENT MIXING SYSTEM WITH PUNCTURE CONNECTION AND MULTIFUNCTIONAL OPERATION

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/075,419

(22) Filed: Mar. 10, 2025

(30) Foreign Application Priority Data
Mar. 15, 2024 (EP) 24163712.3

(51) Int. Cl. A61B 17/88

(52) U.S. Cl. CPC A61B 17/8833; A61B 17/8822; A61B 2017/8838

(57) ABSTRACT

A device for producing bone cement, comprising a base part, which comprises a container having a carrier for receiving a vessel containing monomer liquid, a bottom part having a collection region, a hollow mandrel having a tip, and a piston having a passage for receiving the hollow mandrel, and a cartridge for receiving a PMMA bone cement powder, wherein the cartridge comprises a septum which delimits the cartridge toward the outside. The device further comprises an operating element, and wherein the operating element is configured to open a vessel containing monomer liquid and subsequently to pump a monomer liquid released from the vessel from the collection region, through the hollow mandrel and into the cartridge by moving the piston.

### CROSS-REFERENCE TO RELATED APPLICATIONS

This application claims priority pursuant to 35 U.S.C. 119(a) to European Patent Application No. 24163712.3, filed Mar. 15, 2024, which application is incorporated herein by reference in its entirety.

### FIELD OF THE INVENTION

The present invention relates to the field of medical engineering, in particular to devices for producing a PMMA bone cement and for storing components thereof.

### PRIOR ART

Polymethyl methacrylate bone cements stem from the fundamental work of Charnley. Polymethyl methacrylate bone cements are usually composed of a liquid monomer component and a powder component. The monomer component can, for example, contain the monomer methyl methacrylate and an activator (e.g. N,N-dimethyl-p-toluidine) dissolved therein. The powder component, also referred to as bone cement powder, can comprise one or more polymers producible on the basis of methyl methacrylate and comonomers, such as styrene, methyl acrylate or similar monomers, by polymerization, preferably suspension polymerization. The powder component may further comprise a radiopaque agent and an initiator, such as dibenzoyl peroxide. When the powder component is mixed with the monomer component, a plastically deformable dough, the actual bone cement dough, can form as a result of the polymers of the powder component swelling in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough increases until the cement dough solidifies.

Polymethyl methacrylate bone cements can be mixed by mixing the powder component with the monomer liquid in suitable mixing cups using spatulas. Air bubbles may be incorporated in the bone cement dough, which can negatively influence the mechanical properties of the hardened bone cement.

In order to avoid air inclusions in the bone cement dough, a plurality of vacuum cementing systems have been described, of which the following are mentioned by way of example: U.S. Pat. Nos. 6,033,105 A, 5,624,184 A, 4,671,263 A, 4,973,168 A, 5,100,241 A, WO 99/67015 A1, EP 1 020 167 A2, U.S. Pat. No. 5,586,821 A, EP 1 016 452 A2, DE 36 40 279 A1, WO 94/26403 A1, EP 1 005 901 A2, and U.S. Pat. No. 5,344,232 A.

Cementing systems in which both the cement powder and the monomer liquid are already packed in separate compartments of the mixing systems and are not mixed together in the cementing system until immediately before the cement is applied represent a development in cementing technology. Such closed “fully prepacked mixing systems” are described in patent documents EP0380867B1, EP0796653B1, EP0692229B1, U.S. Pat. Nos. 5,997,544A, 6,709,149B1, DE69812726T2 and U.S. Pat. No. 5,588,745A, WO9416951A1, DE19718648A1, EP1741413B1, EP3054880B1, DE102009031178B3, U.S. Pat. No. 8,662,736B2, EP2269718B1, EP2281532B1 and EP3093067B1.

Fully prepacked mixing systems encounter a few challenges. It is desirable to ensure safe, reproducible transfer of the monomer liquid to the polymethyl methacrylate bone cement powder and to avoid uncontrolled mixing of the components while they are being stored. It is advantageous if the cement powder cannot enter the line means for transferring the monomer liquid during transport and storage of the mixing system. Otherwise, the line means may become blocked when the monomer is transferred as a result of swelling and adhesion. Blockage of the line means by swollen bone cement adhering could interrupt the transfer of the monomer liquid and prevent the cement components from being mixed in the predetermined mixing ratio.

In the fully prepacked mixing systems according to EP0796653B1, U.S. Pat. No. 6,709,149B1, EP1741413B1, EP3054880B1, EP2269718B1 and EP2281532B1, the monomer is transferred by applying an external vacuum to the cartridge via a vacuum connection and a porous plate. This means that the monomer liquid is drawn into the cartridge by the vacuum applied. In the mixing system described in EP3093067B1, the monomer liquid can either be transferred to the cartridge by the action of a vacuum, or alternatively the monomer liquid can be transferred by being pressed by means of a manually operable pump piston integrated into the mixing system.

According to EP2281532B1, the line means for transferring the monomer liquid is prevented from being blocked by using a mesh permeable to the monomer liquid or a porous disk to block the cement powder particles, wherein the mesh or porous disk is part of a special nozzle that generates a sharp jet of monomer liquid when transferring it, which jet displaces swollen cement particles so that all the monomer liquid can be transferred to the bone cement powder.

EP1741413B1 discloses a method for bringing a powder and a liquid component into contact, wherein a liquid container is opened by penetrating a cannula into a wall portion of the liquid container that closes a lateral hole in an inner tube element in the liquid container.

Patent document EP3636338B1 discloses a mixing system comprising a connecting cylinder configured to be selectively coupled to and removable from a mixing chamber, and wherein a valve is configured to seal the connection between the connecting cylinder and the valve when they are selectively coupled to one another, and is configured to seal the mixing chamber when the connecting cylinder is removed from the mixing chamber. EP3490700B1 discloses a similar mixing system.

### PREFERRED EMBODIMENTS

An object of the present invention is to solve one or more of the problems described above and further problems of the prior art. For example, the invention provides a device for storing and mixing bone cement, which allows for easy and reliable mixing of a monomer liquid with a powder component of a PMMA bone cement to produce a ready-to-use PMMA bone cement dough. The devices according to the invention can comprise a plurality of parts which can be easily connected to one another and separated again, thereby allowing for reliable and safe transfer of a monomer liquid from one part of the device to the other part of the device. A self-sealing connection system can prevent monomer liquid from escaping or contamination from entering. The device according to the invention can offer easy handling. A monomer liquid and a powder component of a PMMA bone cement can be separately stored, combined in a controlled manner and mixed together in the device. The device can release a monomer liquid from a closed vessel in an interior of the device and transfer it to another part of the device by means of a movable piston. The device can perform these functions while securely placed on a support. The monomer liquid can be transferred in full, thereby preventing the device from being clogged with clumps of powder.

These objects are achieved by the methods, devices, kits and medical uses described herein, in particular those described in the claims.

Preferred embodiments of the invention will be described below.

A first embodiment relates to a device for producing bone cement, comprising a base part which comprises a container having a carrier for receiving a vessel containing monomer liquid, a bottom part having a collection region, a hollow mandrel having a tip and a piston having a passage for receiving the hollow mandrel, and a cartridge for receiving a PMMA bone cement powder, wherein the cartridge comprises a septum which delimits the cartridge toward the outside;
wherein the device further comprises an operating element, and wherein the operating element is configured to open a vessel containing monomer liquid and subsequently to pump a monomer liquid released from the vessel from the collection region, through the hollow mandrel and into the cartridge by moving the piston.

A second embodiment relates to a device according to the first embodiment, wherein the operating element is movably arranged on the base part.

A third embodiment relates to a device according to the first or second embodiment, wherein the operating element is movable along, and preferably pivotable about, an axis.

A fourth embodiment relates to a device according to any one of the preceding embodiments, wherein, in a first arrangement, the operating element is designed to exert a force on the vessel containing monomer liquid and to thereby prevent the piston from being inserted into the collection region.

A fifth embodiment relates to a device according to any one of the preceding embodiments, wherein, in a second arrangement, the operating element is configured to enable the piston to be inserted into the collection region in order to pump a monomer liquid from the collection region, through the hollow mandrel and into the cartridge.

A sixth embodiment relates to a device according to any one of the preceding embodiments, further comprising an opening element, wherein the operating element is operatively connected or connectable to the opening element, wherein the opening element is designed and configured to open a vessel containing monomer liquid.

A seventh embodiment relates to a device according to any one of the preceding embodiments, wherein the operating element is operatively connected or connectable to the piston.

An eighth embodiment relates to a device according to the seventh embodiment, wherein the operating element comprises a rotary joint.

A ninth embodiment relates to a device according to any one of the preceding embodiments, wherein the device is designed and configured to form a fluid-conducting connection between the base part and the cartridge by piercing the septum with the hollow mandrel in order to transfer a monomer liquid from the collection region to the cartridge.

A tenth embodiment relates to a device according to any one of the preceding embodiments, wherein the cartridge is detachably connectable to the base part.

An eleventh embodiment relates to a device according to any one of the preceding embodiments, wherein the piston is movable by inserting the cartridge into the base part.

A twelfth embodiment relates to a device according to any one of the preceding embodiments, wherein the base part comprises a stop to limit the movement of the piston in the base part, wherein the operating element is preferably designed to engage in the stop.

A thirteenth embodiment relates to a device according to any one of the preceding embodiments, wherein the base part comprises a connecting channel comprising an exit opening, wherein the connecting channel forms a fluid-conducting connection between the container and the collection region.

A fourteenth embodiment relates to a device according to the thirteenth embodiment, wherein the piston is designed and configured to close the exit opening in the connecting channel in a liquid-tight manner when the piston is inserted into the base part.

A fifteenth embodiment relates to a device according to any one of the preceding embodiments, wherein the device is designed and configured to transfer a monomer liquid from the base part to the cartridge while the base part is placed on a support such that the bottom part is oriented toward the support.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 shows the cross section of the base part of a device according to the invention;

FIG. 2 shows the cross section of a device according to the invention, wherein a base part is brought into contact with a cartridge;

FIG. 3 shows the cross section of a device according to the invention, wherein a cartridge is placed on a base part in order to pierce a septum of the cartridge with a hollow mandrel of the base part;

FIG. 4 shows the cross section of a device according to the invention, wherein the operating element exerts a force on the opening element so that a glass ampule is opened in the container;

FIG. 5 shows the cross section of a device according to the invention, wherein a cartridge is placed on a base part, wherein the septum of the cartridge is pierced by a hollow mandrel of the base part;

FIG. 6 shows the cross section of a device according to the invention, wherein a piston is inserted into the base part in order to close an exit opening of a connecting channel in a liquid-tight manner;

FIG. 7 shows a cross section of a cartridge having a mixing rod for mixing a monomer liquid with a PMMA bone cement powder in an interior of the cartridge; and,

FIG. 8 shows a cross section of a cartridge having a threaded rod and a discharge tube for dispensing bone cement dough from the cartridge.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

“Operatively connected” or “operatively connectable” means herein that two elements appertaining thereto have a functional relationship to one another. For example, a first element may be configured to control or move a second element through such an operative connection. The term “control” here also includes blocking or releasing a function, for example enabling or restricting the movement or other function of an element.

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, all the features described herein in connection with a method are in principle also considered to be applicable to the products, devices, kits and uses described herein, and vice versa. All such considered combinations are not explicitly listed in all instances, simply in order to keep the description brief. Technical solutions known to be equivalent to the features described herein are also intended in principle to be encompassed by the scope of the invention.

A first embodiment relates to a device for producing bone cement, comprising a base part which comprises a container having a carrier for receiving a vessel containing monomer liquid, a bottom part having a collection region, a hollow mandrel having a tip and a piston having a passage for receiving the hollow mandrel, and a cartridge for receiving a PMMA bone cement powder, wherein the cartridge comprises a septum which delimits the cartridge toward the outside;
wherein the device further comprises an operating element, and wherein the operating element is configured to open a vessel containing monomer liquid and subsequently to pump a monomer liquid released from the vessel from the collection region, through the hollow mandrel and into the cartridge by moving the piston.

The device according to the invention can have two parts that can be connected to one another, specifically a base part and a cartridge. The base part comprises a container for receiving a monomer liquid. The container comprises a carrier for receiving a vessel containing monomer liquid, for example a glass ampule or a pouch. Such glass ampules or pouches are used as commercially available packaging for monomer liquid of a PMMA bone cement. The container can be prefilled with monomer liquid, for example packaged in a gas-tight manner in a glass ampule or pouch. Alternatively, the container may be empty and fillable with monomer liquid by the user, for example by inserting a glass ampule containing monomer liquid into the container. The cartridge can also be empty or pre-filled with PMMA bone cement powder. The base part may have a transparent region. For example, the container may have a transparent region. The collection region may also have a transparent region. This may allow a user of the device to visually verify that all the monomer liquid has drained from the container into the collection region from the outside. Furthermore, the base part can have a fill level marking. Such a fill level marking can, for example, be arranged at the position of a desired liquid level of the collection region, which level can be achieved by transferring a monomer liquid from the container to the collection region in full.

The device has an operating element. The operating element is configured to open a vessel containing monomer liquid. For example, the operating element can be configured to directly or indirectly exert a force on a vessel containing monomer liquid in order to break it open and release a monomer liquid from the vessel. The operating element is preferably configured to open a vessel containing monomer liquid inside the container of the device.

The operating element is designed to pump a monomer liquid released from the vessel from the collection region, through the hollow mandrel and into the cartridge by moving the piston. This means that, depending on the arrangement or other configuration of the operating element, a pumping function of the device which transfers a monomer liquid from the collection region into the cartridge is enabled. The operating element can therefore, for example, enable the pumping function of the piston by releasing the piston. Alternatively or additionally, the operating element can exert a force on the piston to operate the pumping function of the piston.

The operating element is therefore configured to open a vessel containing monomer liquid and subsequently to pump a monomer liquid released from the vessel from the collection region, through the hollow mandrel and into the cartridge by moving the piston.

The operating element can be movably arranged on the base part. The operating element can be movable along an axis. This axis can be parallel to a direction of movement of the piston. The axis can extend in the direction of a bottom part of the base part, in particular in the direction of a supporting surface of the bottom part. This allows the operating element to be inserted into the base part, at least in portions. The operating element can also be pivotable about such an axis. This allows the operating element to be moved from a first arrangement to a second arrangement. For example, the operating element can have a rotary joint for this purpose. The rotary joint can, for example, be designed as a ring-shaped holder which engages in a cylindrical housing part of the base part. The control element may have a handle that is designed and configured to allow a user to move the operating element. The handle may have a flat surface to enable a user to partially move the operating element into or toward the base part with one hand, for example with a finger.

In a first arrangement, the operating element can be configured to exert a force on a vessel containing monomer liquid, thereby preventing the entire piston from being inserted into the collection region. This allows a vessel containing monomer liquid, such as a glass ampule or a foil pouch, to be opened. The monomer liquid can then pass from the opened vessel into the collection region, preferably by means of free gravity flow. In the first arrangement, the device is preferably designed to hold a monomer liquid in the collection region. In the first arrangement, the monomer liquid is preferably not transferred to the cartridge. This allows a temporal separation between the release of the monomer liquid from the vessel on the one hand and the transfer of the monomer liquid to the cartridge on the other.

In a second arrangement, the operating element can be designed to enable the piston to be inserted into the collection region. This allows a monomer liquid to be pumped from the collection region, through the hollow mandrel and into the cartridge.

As explained above, the operating element can preferably be moved from a first arrangement to a second arrangement by being pivoted about an axis. In a similar way, other technical solutions are also possible for moving the operating element from a first arrangement to a second arrangement. This makes it possible to select the function of the operating element, according to which the operating element is designed either to open a vessel containing monomer liquid or to pump a monomer liquid from the collection region into the cartridge, depending on the arrangement of the operating element.

The device may further comprise an opening element. The opening element is preferably designed and configured to open a vessel containing monomer liquid. The operating element is preferably operatively connected or connectable to the opening element. Examples of an opening element include a pin or wedge that can be pressed against the head of a glass ampule to break the head off the glass ampule. For example, the operating element can be configured to transmit a force via the opening element to a vessel containing monomer liquid in order to open the vessel and release monomer liquid therefrom. The operating element is preferably arranged so that it can engage in the opening element. The opening element can be functionally connected to the piston. The piston can be designed and configured to move the opening element.

The opening element is preferably configured to open a foil pouch or a glass ampule inside the device. An example of an opening element for opening a foil pouch is a mandrel, which is suitable for piercing a foil pouch. Such a mandrel can be made, for example, from a rigid plastics material or from metal. Another example of an opening element for opening a glass ampule is a projection, against which the head of a glass ampule can be pressed to break it open. A means for opening a glass ampule can also have a movably arranged element, such as a piston, which can press the glass ampule against a hard housing part, e.g. a projection, and thereby break it open. Opening elements for opening a foil pouch or a glass ampule are further disclosed in DE19532015A1, WO9718031A1, EP2404864B1 and WO2010012114A1.

In one embodiment, the container has a projection arranged in the container, which is designed and configured to break open a glass ampule that can be received in the container, wherein the operating element is designed and configured to break open a glass ampule that can be received in the holder by pressing the glass ampule against the projection.

In one embodiment, the base part comprises a stop. The stop may be designed and configured to limit the movement of the piston in the base part.

The operating element can be designed to engage in the stop. In a first arrangement described herein, the operating element can in particular be configured to engage in the stop. This can prevent monomer liquid from being pumped from the collection region into the cartridge in a first arrangement of the operating element. The stop can, for example, be arranged on an outer side of the base part. The stop can be designed as a projection which extends in the direction of the operating element.

The base part can be designed to be placed on a support. The base part has a bottom part which can have a flat, preferably planar, outer side. The bottom part has a collection region. The collection region is preferably designed and configured to receive a monomer liquid. For example, the base part can be configured to release monomer liquid from a vessel, such as a glass ampule or a foil pouch, and to transfer it to the collection region by gravity flow. The collection region may have a recess, for example concave curvature, similar to a watch glass. The cross section of the recess can be U shaped or V shaped. The recess may be funnel-shaped.

The collection region preferably has a capacity for receiving monomer liquid that exceeds the volume of monomer liquid contained or receivable in the container. For example, the collection region may have a volume which is at least 1.1 times the volume of the monomer liquid contained or receivable in the container. For example, if the container has a carrier for receiving commercially available glass ampules containing 10 ml of monomer liquid, the collection region preferably has a volume of at least 11 ml. This applies, mutatis mutandis, to embodiments which comprise a carrier for receiving glass ampules with 5 ml, 20 ml or 30 ml of monomer liquid.

The base part also has a hollow mandrel having a tip. The hollow mandrel preferably has an interior that allows a liquid to flow through the hollow mandrel.

The interior can have a diameter of 1.0 mm to 3.0 mm. The hollow mandrel can have outlet openings with a diameter of less than 1.5 mm, and particularly preferably less than 1.0 mm. As a result of the hollow mandrel having a small diameter, a sharp jet can be formed when a monomer liquid is transferred from the collection region into the cartridge, which jet can optionally displace smaller particles of bone cement powder. In this way, clogging of the hollow mandrel can be avoided. The hollow mandrel can have a beveled tip, similar to a ground cannula, with an outlet opening of the hollow mandrel preferably being located in a bevel of the tip. The hollow mandrel can have a round tip. One or more openings can be arranged near the tip, which are connected to the interior of the hollow mandrel. Furthermore, the interior of the cavity can have a shoulder on which a porous pin permeable to liquids is supported. This pin can serve as a filter, as described hereinbelow. The interior of the hollow mandrel can taper toward the shoulder. The shoulder may be configured to hold the pin in the hollow mandrel. The hollow mandrel can be made of plastics material or metal, e.g. stainless steel. Examples of usable plastics include Nylon 12, Nylon 6, Nylon 66 and polyethersulfone. The hollow mandrel, particularly the tip of the hollow mandrel, may be covered by a removable protective cap.

The cartridge comprises a septum which delimits the cartridge toward the outside. The septum is preferably arranged in a region of the cartridge which is designed and configured for connection to the base part. The septum can be arranged in a discharge piston or in a cartridge head of the cartridge. The septum can be pierced by the hollow mandrel of the base part. The septum may have a perforation which facilitates piercing of the septum in a predetermined position on the septum. In a configuration of the device in which the septum is pierced by the hollow mandrel, a fluid-conducting connection can form between the base part and the cartridge. This makes it possible to transfer a monomer liquid from the collection region of the base part to the cartridge. The self-sealing function of the septum can prevent monomer liquid from escaping from the device. This also applies if the hollow mandrel is pulled back out of the septum, as the puncture site can close automatically due to the restoring force of the septum. Furthermore, this can prevent contamination from entering the device from the outside. In one embodiment, the device is designed and configured to form a fluid-conducting connection between the base part and the cartridge by piercing the septum with the hollow mandrel in order to transfer a monomer liquid from the collection region to the cartridge.

The term “fluid-conducting connection” or “liquid-conducting connection” means here that a monomer liquid can flow from one part of the device to another part of the device.

The septum can comprise a rubber-elastic material. In some embodiments, the septum comprises a flexible, self-sealing polymer. The polymer may comprise a polyhalogenated olefin, a silicone, rubber, butyl rubber, ethylene propylene diene rubber (EPDM), or a similar elastomer. The septum is preferably designed to close the cartridge in a liquid-tight manner after the septum has been pierced by the hollow mandrel. The septum is preferably stretchable and exhibits rubber-elastic properties, as is known, for example, from septa in chemical bottles. This allows the septum to heal itself after being pierced, i.e. to automatically seal.

The septum preferably comprises a biocompatible material. The septum can comprise a circular shape. In one embodiment, the septum comprises a material which can be pierced with a medical injection cannula according to ISO 7864:2016 with a force of less than 100 N. The septum is preferably connected to the cartridge in a liquid-tight manner. In one embodiment, the septum is designed and configured to close a puncture site in the septum in a liquid-tight manner after the septum has been punctured and the hollow mandrel has subsequently been removed. This can be achieved by the restoring force of a rubber-elastic material of the septum.

The cartridge may have a guide element, e.g. an insertion bevel, which facilitates insertion of the hollow mandrel into a desired position in the septum.

The septum can be mounted in a holder. The holder can be detachably connected to the cartridge. The septum holder can be designed as a piston. This piston may be designed and configured to dispense bone cement dough from the cartridge. The septum holder can be arranged in a cartridge head. The cartridge head can be designed as a releasable cartridge closure.

The cartridge and the base part can preferably be mounted separately from one another, i.e. they are designed as separate sub-elements that can be detachably connected to one another. This can prevent fatigue of the septum's restoring force when mounted and facilitate the automatic closure of the septum. Unwanted escape of a monomer liquid from the cartridge after removing the hollow mandrel from the septum can be prevented. Furthermore, separate mounting has the advantage that packaging materials with small dimensions can be used.

The device comprises a piston. The piston can be arranged inside the base part. The piston may have a passage for receiving the hollow mandrel. The passage can extend from an interior of the base part to the outside. The piston may be configured to receive the tip of the hollow mandrel in the passage in a basic configuration of the device. In such a basic configuration of the device, no external forces are exerted on the device. In this state, the tip of the hollow mandrel is preferably arranged inside the piston so that a user is protected from injury as a result of contact with the tip. In one embodiment, the piston is configured to release the tip of the hollow mandrel from the passage when the cartridge is pressed onto the piston. This allows the cartridge to apply pressure to the piston to release the hollow mandrel and pierce the septum of the cartridge. The tip of the hollow mandrel can protrude from the passage. This thus allows the cartridge and base part to be connected to one another in a fluid-conducting manner in a way that prevents injury in order to receive a monomer liquid in the cartridge. The piston is preferably configured to be inserted through the cartridge into the base part when the cartridge is connected to the base part. The piston can therefore be moved by the cartridge exerting pressure against the piston.

In some embodiments, the tip of the hollow mandrel is protected by a removable cover.

According to the invention, the piston is designed to pump a monomer liquid from the collection region, through the hollow mandrel and into the cartridge. For this purpose, the piston may be configured to increase the pressure within the collection region so that the pressure within the collection region exceeds the pressure in the cartridge. In some embodiments, the device is designed and configured to enable a monomer liquid to be transferred to the cartridge independently of an external vacuum source.

In some embodiments, the piston is movably arranged in the base part. By applying pressure to the piston, the piston can preferably be movable in the direction of the collection region of the base part. In this case, the piston can be moved in the direction of the interior of the base part or in the direction of a supporting surface of the base part. This movement can increase the pressure in the collection region to pump a monomer fluid from the collection region, through the hollow mandrel and into the cartridge.

Preferably, the operating element is operatively connected or connectable to the piston. The device can in particular be designed to control the function of the piston on the basis of the arrangement of the operating element. As described herein, the pumping function may be disabled in a first arrangement of the operating element, while the pumping function may be enabled in a second arrangement of the control element.

In some embodiments, the piston is designed and configured to be fully inserted into the collection region so that no dead volume remains in the collection region. For this purpose, a side of the piston. which is oriented toward the collection region. can have a surface shape which complements a surface shape of the collection region. For example, the piston may have a convex front side and the collection region may have a concave surface. The piston may have a raised portion that complements a recess in the collection region. This allows substantially all the monomer liquid to be transferred from the collection region to the cartridge, since only a small dead volume remains within the base part (particularly within the hollow mandrel) when the monomer liquid is pumped from the base part into the cartridge by the movement of the piston.

In some embodiments, the device is designed and configured to simultaneously establish a fluid-conducting connection between the base part and the cartridge and to open a vessel containing a monomer liquid. The cartridge can exert pressure on the piston to release the hollow mandrel from the passage and pierce the septum, while an opening element opens the vessel at the same time. For this purpose, the piston can be operatively connected to the opening element.

In some embodiments, the device is designed and configured to open a vessel containing a monomer liquid by a first movement of the piston and to transfer a monomer liquid from the collection region to the cartridge by a second movement of the piston, wherein the second movement is enabled by the transition of the operating element from a first arrangement of the operating element to a second arrangement of the operating element.

The piston may have a releasable safety device which prevents unintentional movement of the piston, for example during transport or any other period when the device is not in use. This safety device can, for example, be designed as an open clamping ring. This can prevent a vessel containing monomer liquid from unintentionally opening in the device.

In one embodiment, the base part has a spring which is arranged and configured for movably mounting the piston within the base part. This allows the piston to be held in a position in which the tip of the hollow mandrel is received in the passage of the piston, as described above, and is thus held within the piston in a way that prevents injury. The spring can be a spiral spring. In one embodiment, the spring surrounds the hollow mandrel in a portion of the hollow mandrel that is located outside the passage of the piston. The spring can be arranged between the bottom part and the piston.

In an embodiment in which the hollow mandrel is mounted in a cylindrical holder, as described herein, the spring can be arranged between this holder and the piston. The spring can facilitate the separation of the base part and the cartridge. Furthermore, the tip of the hollow mandrel can be automatically moved back into the cavity by means of the spring as soon as no external forces are acting on the piston, e.g. when the user no longer actively presses the cartridge against the base part. The spring can also be designed to hold the piston in a position in which the connecting channel is open, i.e. wherein the connecting channel forms a fluid-conducting connection between the container and the collection region, and the piston does not sealingly close the exit opening in the connecting channel.

In one embodiment, the base part comprises a connecting channel with an exit opening. The connecting channel can form a fluid-conducting connection between the container and the collection region. Thus, a monomer liquid can be transferred from the container to the collection region via the connecting channel. The connecting channel preferably has an exit opening, wherein the exit opening is preferably arranged at an end of the connecting channel which points in the direction of the collecting region. Preferably, the connecting channel is designed and configured to transfer monomer liquid from the container to the collection region by means of free gravitational flow. This applies in particular to an arrangement of the device in which the base part is placed on a support so that the bottom part is oriented toward the support, as described herein. “Free gravitational flow” means here that a monomer liquid can pass from the container into the collection region by the effect of gravity alone, without the need for, for example, a pressure difference between the container and the collection region. Preferably, the devices described herein allow all of the monomer liquid to be transferred from the container to the collection region by means of free gravitational flow within 20 seconds. The monomer liquid can then be transferred from the collection region to the cartridge by being pumped by means of the piston, as described herein.

In one embodiment, the piston is designed and configured to close the exit opening in the connecting channel in a liquid-tight manner when the piston is inserted into the base part. As a result of the piston moving in the direction of the collection region, a lateral surface of the piston can be brought into contact with the exit opening in the connecting channel to close the exit opening. In such an arrangement of the device, a fluid-conducting connection does not exist between the container and the collection region. In this configuration, the collection region is sealed with respect to the container by the piston such that there is only a fluid-conducting connection between the collection region and the hollow mandrel. This allows the piston to pump monomer liquid from the collection region through the hollow mandrel, since in this arrangement pressure equalization with the container cannot occur via the connecting channel.

The device is preferably designed to release the exit opening in a first arrangement of the operating element and to close the exit opening by means of the piston in a second arrangement of the operating element.

The exit opening can have a cross-sectional area of 50 mm², preferably at least 100 mm² and particularly preferably greater than 100 mm².

Depending on the arrangement of the operating element, the position of the piston can be held above or below the exit opening according to whether the exit opening is open or closed. Accordingly, the device according to the invention can be designed and configured to dispense monomer liquid from the container into the collection region in a first arrangement of the operating element described herein, and to pump monomer liquid from the collection region through the hollow mandrel in a second arrangement of the operating element described herein. The hollow mandrel can be used to pierce the septum of the cartridge in order to transfer monomer liquid from the base part to the cartridge.

In one embodiment, the device comprises a filter. The filter can be arranged in the base part. For example, the filter can be arranged between the container and the hollow mandrel. The filter can be arranged between the container and the connecting channel. The filter is preferably designed and arranged to prevent particles from the base part from entering the cartridge. For example, the filter may be arranged and configured to retain glass fragments from a glass ampule broken open in the base part. The filter can be, for example, a metal wire mesh or a mesh made of a plastics fabric. The filter can also be a disk made of metal or plastics material provided with expediently dimensioned holes. Furthermore, open-pore foams, such as Porex, can be used. Preferably, the filter should be made of a material which is not attacked by the monomer liquid located in an accommodable glass ampule.

In one embodiment, the container is configured to allow the transfer of a monomer liquid from the container to the collection region by means of free gravitational flow.

In one embodiment, the connecting channel is arranged at an angle to the bottom part of from 20° to 80°, preferably 21° to 55°. This angle is defined by the inner side of each container and bottom part where the connecting channel and bottom part merge into one another in the direction of the bottom part (i.e. in the direction of the “lower side” of the base part) at a vertical lateral wall of the bottom part. This corresponds to the point where the monomer liquid flows from the connecting channel into the collection region when the device is used as intended. The container is preferably arranged opposite the bottom part, i.e. oriented “upward” when the device is used as intended, in order to allow the monomer liquid to flow into the collection region. For example, the connecting channel can be arranged relative to the bottom part in a similar way to a wing of a wing nut. The base part can, for example, have two or more containers and connecting channels. In one embodiment, the base part comprises two connecting channels, each of which is arranged in a Y-shape with respect to the bottom part. The piston can be arranged between the containers, i.e. between the two connecting channels which form the “Y arms”.

The container may be arranged so that in a configuration of the base part in which the base part is placed on a support, wherein the bottom part is oriented toward the support, the container is located above the collection region. In particular, the exit opening of the connecting channel can also be arranged above the collection region in such a configuration. This allows for the monomer liquid to flow reliably from the container into the collection region. Furthermore, this enables an arrangement of the piston in which the exit opening in the connecting channel is sealed by the piston, and monomer liquid can be received in the collection region, i.e. sufficient free volume is available in the collection region to accommodate monomer liquid.

In one embodiment, a longitudinal axis of the connecting channel is arranged at an angle of from 20° to 80°, preferably 21° to 55°, to an axis that runs perpendicularly to a support surface of the bottom part. At an angle of 21° to 55°, the formation of a meniscus by the monomer liquid can in particular be prevented.

In one embodiment, the cartridge can be detachably connected to the base part. For this purpose, appropriate connecting elements, such as a thread, can be provided on the cartridge and the base part. The connecting elements can be arranged in such a way that they allow the hollow mandrel to be connected to the septum. Furthermore, the device can have a guide element which facilitates the connection of the base part to the cartridge in a desired orientation. Preferably, the base part and the cartridge can be connected to one another in such a way that the piston and the septum are flush with one another. For this purpose, for example, the base part can have a projecting region which is designed and configured to engage in the cartridge.

In one embodiment, the base part has a line element, wherein the line element is configured to establish a fluid-conducting connection between the collection region and the hollow mandrel. The line element enables monomer liquid to be transferred from the collection region to the hollow mandrel, preferably by free gravitational flow. The line element can be designed, for example, as a hose, tube or passage. In an embodiment in which the hollow mandrel is mounted in a cylindrical holder, as described herein, the line element can be designed as a passage in the holder.

In one embodiment, the device is designed and configured to transfer a monomer liquid from the base part to the cartridge while the base part is placed on a support so that the bottom part is oriented toward the support. The containers and the cartridge preferably point “upward”, i.e. in the opposite direction to the support and against the direction of gravity. The monomer liquid can therefore flow from the container into the collection region as a result of the effect of gravity.

In some embodiments, the cartridge comprises a PMMA bone cement powder. The PMMA bone cement powder is preferably arranged in an interior of the cartridge.

In some embodiments, the container comprises a monomer liquid. The monomer liquid is preferably arranged in an interior of the container. The monomer liquid may be arranged in a vessel, such as a glass ampule or in a sealed foil pouch, within the container.

In some embodiments, the container further comprises a filter permeable to a monomer liquid. The filter is preferably arranged and designed to prevent glass splinters from the container from entering the cartridge. The filter can, for example, be arranged between the carrier and the connecting channel.

The device can be designed to receive a glass ampule containing monomer liquid. The device can further be designed to break open a glass ampule containing monomer liquid. It is possible that the glass ampule can be received and broken open, in particular within the container. For this purpose, the container can have a carrier, e.g., a receptacle for a glass ampule. The container may comprise a means for breaking open a glass ampule. For example, the container may have a projection arranged and configured to break off the head of a glass ampule. The container may have a catch region for broken glass. The filter can be arranged between this catch region and the connecting channel.

The filter may be designed to retain fragments of a glass ampule in the container. This can prevent glass splinters from entering the cartridge. This can prevent contamination of bone cement dough with glass splinters.

The monomer liquid filters described herein may be arranged either in the container or at another location on the base part, or both. For example, the container may comprise a first filter, and the line element connecting the collection region to the hollow mandrel may comprise a second filter. The second filter may have a smaller void volume than the first filter.

In some embodiments, the device further comprises a mixing rod. The mixing rod is preferably designed for mixing monomer liquid and PMMA bone cement powder. The mixing rod may have blades. The blades can facilitate homogeneous mixing of monomer liquid and PMMA bone cement powder. The blades are preferably arranged at an end of the mixing rod which is arranged inside the cartridge. The blades can be arranged on a disk of the mixing rod. The mixing rod can have one, two or more than two blades. The mixing rod may have a handle at an end of the mixing rod that is preferably arranged outside the cartridge. The mixing rod is preferably movably arranged in the cartridge.

The mixing rod can comprise a discharge tube. Here, the discharge tube can be designed and configured to dispense a bone cement dough mixed in the device (i.e. a bone cement dough that has been mixed from monomer liquid and PMMA bone cement powder). Preferably, the discharge tube is closable. This can prevent bone cement dough from undesirably escaping from the cartridge during mixing. The discharge tube can be designed as a cavity within the mixing rod. In some embodiments, the discharge tube can be closed by a removable core. Accordingly, the mixing rod can have a detachable, rod-shaped core, for example a metal core. The core can also improve the strength of the mixing rod.

The mixing rod may have a predetermined breaking point to allow the mixing rod to be broken off after the mixing process. The predetermined breaking point is preferably located outside the cartridge so that the mixing rod can be broken off from the outside without opening the cartridge.

The cartridge may have a connection for a vacuum source, e.g. a pump. This allows monomer liquid and PMMA bone cement powder in the cartridge to be mixed under vacuum conditions.

Furthermore, the base part can have a ventilation opening. The ventilation opening can be arranged in the container. The ventilation opening can preferably be arranged in the base part in such a way as to allow for pressure equalization while the piston keeps the exit opening in the connecting channel free, i.e. does not close it. Preferably, the ventilation opening is arranged in the base part in such a way that contact between the ventilation opening and monomer liquid is avoided when the device is used as intended. The ventilation opening can be designed either as a simple opening or as a valve. The opening can be covered on the inside by a gas-permeable porous material to prevent particles from entering the container from the outside and at the same time allowing for air to be exchanged through the porous material. The valve can, for example, be designed to allow air to pass only toward the interior of the container. This can improve the transfer of the monomer liquid into the collection region. Furthermore, a unidirectional valve can prevent monomer liquid from undesirably leaving the device.

A further aspect relates to a method for producing a bone cement dough, wherein the method comprises the following steps:

Providing a device according to any one of the embodiments described herein;

Optionally introducing a vessel containing a monomer liquid into the container;

Optionally introducing a PMMA bone cement powder into the cartridge;

Bringing the cartridge into contact with the base part;

Inserting the hollow mandrel through the septum such that a fluid-conducting connection is formed between the collection region and the cartridge;

Optionally positioning the operating element in a first arrangement which allows the vessel to open;

Opening the vessel using the operating element, wherein the operating element exerts a force on the vessel to open it and to release a monomer liquid therefrom;

Optionally arranging the device so that the monomer liquid is transferred from the container to the collection region by free gravitational flow;

Positioning the operating element in a second arrangement which allows for a pumping process performed by the piston;

Pressing the cartridge against the piston so that monomer liquid is pumped from the container into the cartridge;

Mixing the monomer liquid with the PMMA bone cement powder in the cartridge to obtain a bone cement dough.

A further aspect relates to a method for producing a bone cement dough, wherein the method comprises the following steps:

Providing a device comprising a base part having a vessel containing monomer liquid, a movable piston having a passage, and a hollow mandrel, and a cartridge having a septum and a PMMA bone cement powder, wherein the device further comprises an operating element;

Optionally positioning the operating element in a first arrangement which allows the vessel to open;

Opening the vessel using the operating element, wherein the operating element exerts a force on the vessel to open it and to release a monomer liquid therefrom;

Inserting the hollow mandrel through the septum such that a fluid-conducting connection is formed between the collection region and the cartridge;

Positioning the operating element in a second arrangement which allows for a pumping process performed by the piston;

Pressing the cartridge against the piston so that monomer liquid is pumped from the container into the cartridge;

Mixing the monomer liquid with the PMMA bone cement powder in the cartridge to obtain a bone cement dough.

A further aspect relates to the use of a device described herein or the method described above for producing bone cement dough, in particular PMMA bone cement dough. Here, a monomer liquid can be mixed with a PMMA bone cement powder within the device to form a bone cement dough. Such a bone cement dough can be dispensed from the device and hardened into PMMA bone cement at a target location.

### EXAMPLES

The invention is further illustrated below using examples which are, however, not to be understood as limiting. It will be apparent to a person skilled in the art that other equivalent means may be used similarly in place of the features described here.

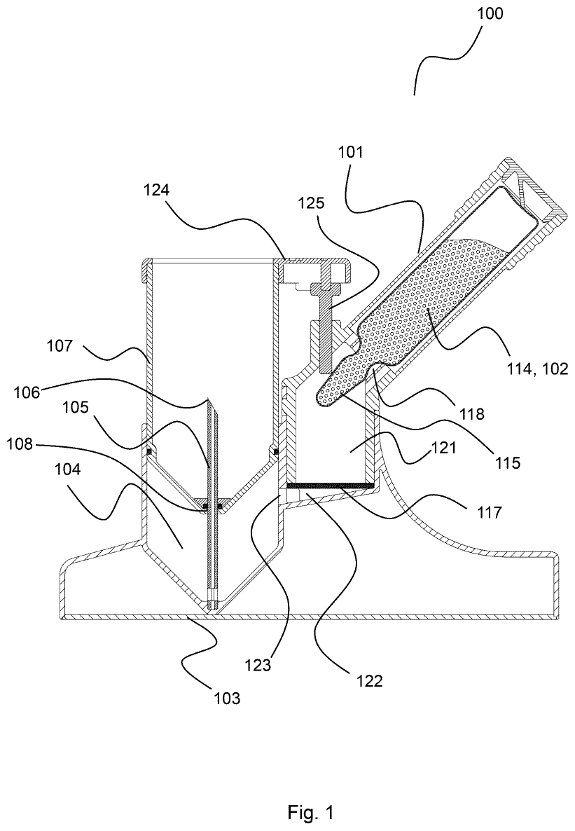

FIG. 1 shows the cross section of the base part of a device according to the invention. The base part 100 comprises a container 101 for receiving a monomer liquid. A carrier 118 which comprises a glass ampule 114 containing monomer liquid 102 is arranged in the container 101. The base part also comprises a bottom part 103 which has a flat supporting surface for placing the base part on a support. On the bottom part is a collection region 104 for receiving a monomer liquid. The collection region has a recess, with a hollow mandrel being arranged at the deepest point of this recess 105. The hollow mandrel is connected to the collection region in a liquid-conducting manner so that monomer fluid can enter the hollow mandrel from the collection region. The container also comprises a catch region 121 for receiving glass fragments, such as a broken ampule head 115 and retain them therein. The container is connected via a connecting channel 122 to the collection region 104 in a liquid-conducting manner. Thus, a monomer liquid 102 can pass from the container 101 into the collection region 104. A filter 117 is arranged between the catch region 121 and the connecting channel 122. By means of the filter 117, glass fragments can be retained in the catch region 121. If the device is placed on a support by means of the base part 103, monomer liquid can flow from the container into the collection region by means of gravitational flow. Therefore, a pressure difference is not required to transfer the monomer liquid from the container to the collection region. The connecting channel 122 comprises an exit opening 123 at an end of the connecting channel that extends toward the collection region 104. The exit opening 123 is arranged above the collection region 104. The base part 100 further comprises a piston 107 having a passage 108. The hollow mandrel 105 can be received in the passage 108. The hollow mandrel 105 can establish a liquid-conducting connection between the collection region 104 and a cartridge 200. In the arrangement shown in this figure, the hollow mandrel 105 passes through the passage 108. The hollow mandrel 105 comprises a tip 106. The base part 100 further comprises an operating element 124 which is movably arranged on the base part 100. In this case, the operating element 124 is shown in a first arrangement in which the operating element 124 is operatively connected to an opening element 125. The operating element 124 engages in the opening element 125 so that the opening element 125 can be inserted into the base part 100 portion-by-portion when a user applies a force to the operating element 124 which can be transferred to the opening element 125. The opening element 125 is configured to open a vessel 114 containing monomer liquid that is received in the container. In the embodiment shown here, the opening element 125 is designed as a pin, which can be pressed against the head 115 of a glass ampule 114 in order to break off the head 115 of the glass ampule 114 and release the monomer liquid 102 contained therein into the base part 100.

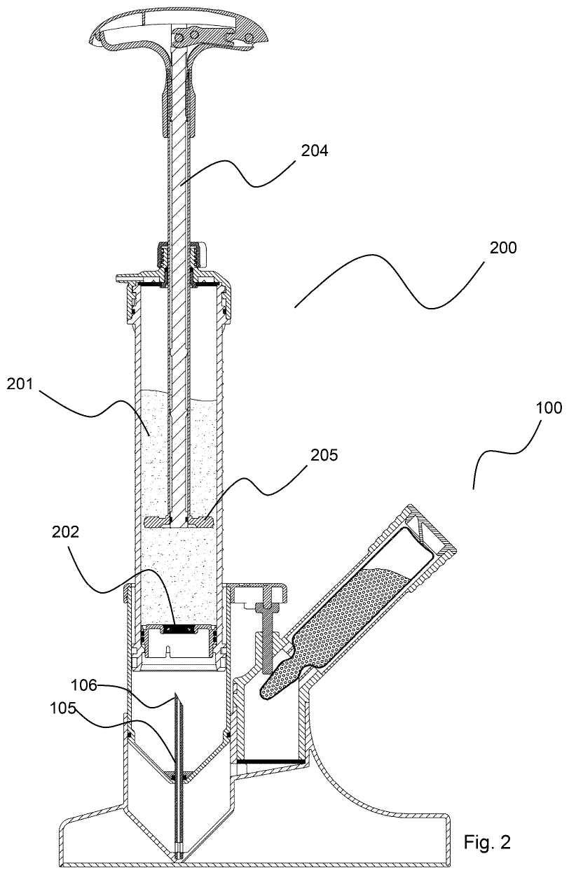

FIG. 2 shows the cross section of a device according to the invention, wherein a base part 100 is brought into contact with a cartridge 200. The cartridge 200 comprises a septum 202 made of a rubber-elastic material, which delimits an interior of the cartridge, which contains a PMMA bone cement powder 201, toward the outside. The cartridge also has a mixing rod 204 comprising a mixing disk 205.

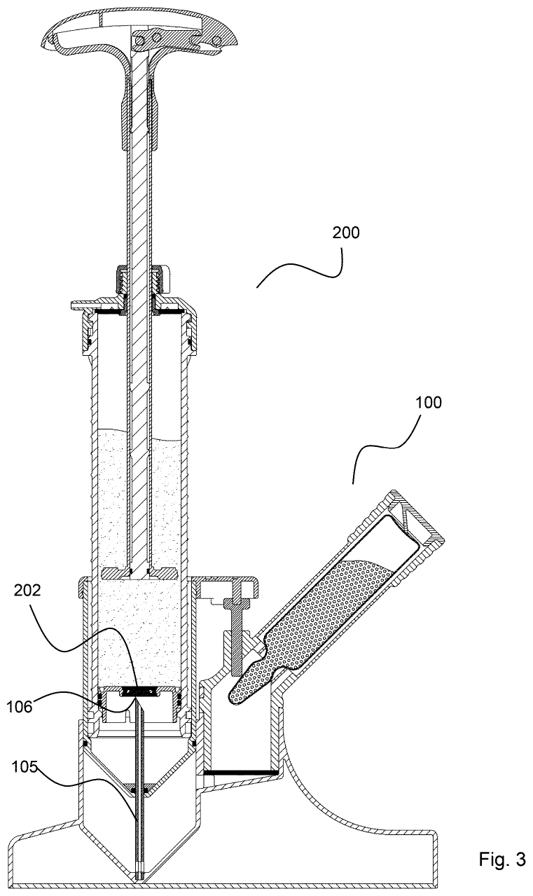

FIG. 3 shows the cross section of a device according to the invention, wherein a cartridge 200 is placed on a base part 100 in order to pierce a septum 202 of the cartridge 200 with a hollow mandrel 105 of the base part. The cartridge 200 is detachably connectable to the base part 100 so that the tip 106 of the hollow mandrel 105 can penetrate the septum 202 and thereby create a liquid-conducting connection between the collection region 104 and an interior of the cartridge 200.

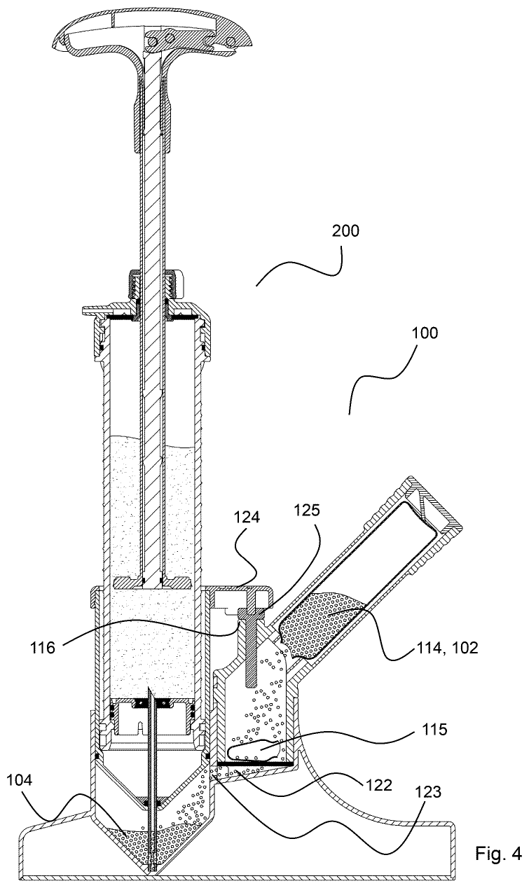

FIG. 4 shows the cross section of a device according to the invention, wherein the operating element 124 exerts a force on the opening element 125 such that a glass ampule 114 is opened in the container 101. In this case, the opening element 125 is designed as a pin which can be pressed against the head 115 of a glass ampule 114. This opens the glass ampule 114 in order to release monomer liquid 102 from the glass ampule.

The monomer liquid 102 can flow into a collection region 104 of the base part 100. The monomer liquid 102 flows via a connecting channel 122 from the glass ampule 114, through an exit opening 123 in the connecting channel 122 and into the collection region 104. A stop 116 engages with the opening element 125, which in turn engages with the operating element 124. This prevents the entire piston 107 from being pressed into the collection region 104, and therefore the pumping function of the piston 107 is blocked in this first arrangement of the operating element 124.

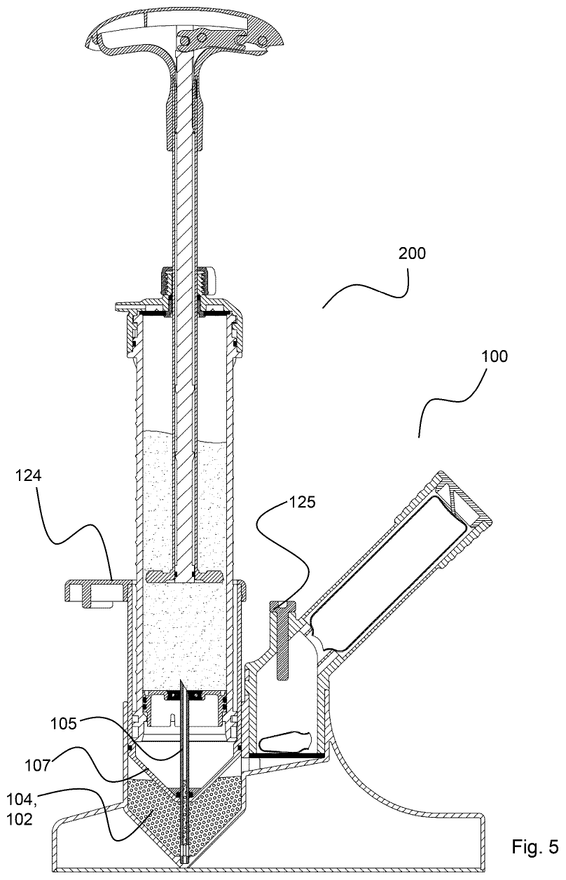

FIG. 5 shows the cross section of a device according to the invention, wherein a cartridge 200 is placed on a base part 100, wherein the septum 202 of the cartridge 200 is pierced by a hollow mandrel 105 of the base part. In the second arrangement of the operating element shown here, the operating element 124 is pivoted to the side so that it no longer engages with the opening element 125. In this second arrangement of the operating element, the pumping function of the piston 107 can be performed. The operating element 124 can exert a force on the piston 107 to completely move it into the collection region 104. This allows monomer liquid 102 to be pumped from the collection region 104, through the hollow mandrel 105 and into the cartridge 200.

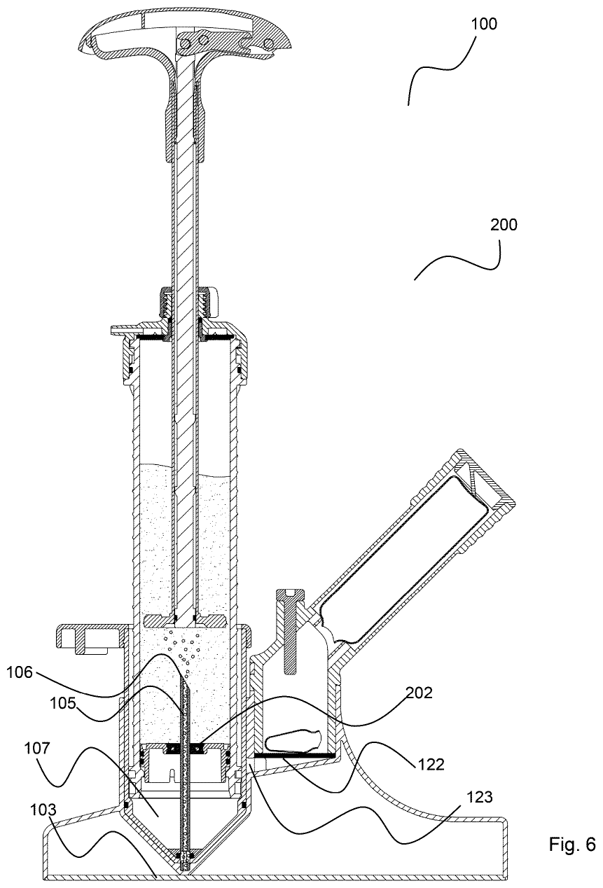

FIG. 6 shows the cross section of a device according to the invention, wherein a piston 107 is inserted into the base part 100 to close an exit opening 123 in a connecting channel 122 in a liquid-tight manner. One side of the piston ends with the exit opening 123 in order to close the exit opening. The tip 106 of the hollow mandrel penetrates the septum 202 such that a liquid-conducting connection is created between the collection region 104 and the interior of the cartridge 200. The piston 107 exerts pressure on the collection region 104 in order to pump monomer liquid 102 from the collection region 104, through the hollow mandrel 105 and into the cartridge 200.

The piston 107 is designed to pump a monomer liquid from the collection region, through the hollow mandrel 105 and into the cartridge 200. The piston 107 is movably arranged in the base part 100. The piston 107 can be inserted into the base part 100 in the direction of the collection region 104 or in the direction of the bottom part 103, thereby exerting pressure on the collection region 104 such that a monomer liquid can pass from the collection region 104 into the cartridge 200 through the hollow mandrel 105.

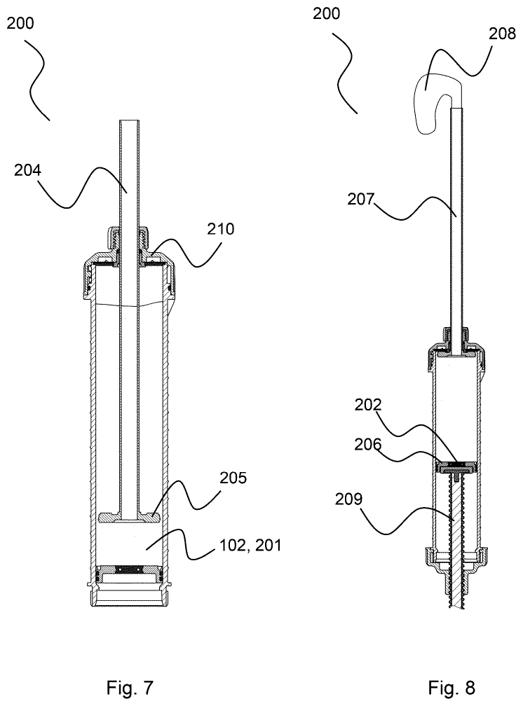

FIG. 7 shows a cross section of a cartridge 200 comprising a mixing rod 204 for mixing a monomer liquid 102 with a PMMA bone cement powder 201 in an interior of the cartridge 200. The mixing rod 204 passes through a cartridge head 210. The mixing rod 204 is movably arranged in the cartridge 200 and has a mixing disk 205 which is arranged inside the cartridge 200 at one end of the mixing rod 204 so as to improve the process of mixing monomer liquid and PMMA bone cement powder in the cartridge 200. By mixing monomer liquid and PMMA bone cement powder, a bone cement dough can be produced in the cartridge.

FIG. 8 shows a cross section of a cartridge 200 having a threaded rod 209 and a discharge tube 207 for dispensing bone cement dough 208 from the cartridge 200. The threaded rod 209 is designed to move a holder 206, which supports the septum 202, inside the cartridge to dispense bone cement dough 208 from the cartridge 200 to the outside via the discharge tube 207.

### LIST OF REFERENCE NUMERALS

100 base part
101 container
102 monomer liquid
103 bottom part
104 collection region
105 hollow mandrel
106 tip
107 piston
108 passage
111 line element
114 vessel
115 ampule head
116 stop
117 filter
118 carrier
119 projection
121 catch region
122 connecting channel
123 exit opening
124 operating element
125 opening element
200 cartridge
201 PMMA bone cement powder
202 septum
204 mixing rod
205 mixing disk
206 septum holder
207 discharge tube
208 bone cement dough
209 threaded rod
210 cartridge head

What is claimed is:

1. A device for producing bone cement, comprising
a base part, which comprises a container having a carrier for receiving a vessel containing monomer liquid, a bottom part having a collection region, a hollow mandrel having a tip, and a piston having a passage for receiving the hollow mandrel, and,
a cartridge for receiving a PMMA bone cement powder, wherein the cartridge comprises a septum which delimits the cartridge toward the outside; wherein the device further comprises an operating element, and wherein the operating element is configured to open a vessel containing monomer liquid and subsequently to pump a monomer liquid released from the vessel from the collection region, through the hollow mandrel and into the cartridge by moving the piston.

2. The device according to claim 1, wherein the operating element is movably arranged on the base part.

3. The device according to claim 1, wherein the operating element is movable along, and preferably pivotable about, an axis.

4. The device according to claim 1, wherein, in a first arrangement, the operating element is designed to exert a force on a vessel containing monomer liquid, thereby preventing the piston from being fully inserted into the collection region.

5. The device according to claim 1, wherein, in a second arrangement, the operating element is designed to enable the piston to be inserted into the collection region in order to pump a monomer liquid from the collection region, through the hollow mandrel and into the cartridge.

6. The device according to claim 1, further comprising an opening element, wherein the operating element is operatively connected or connectable to the opening element, wherein the opening element is designed and configured to open a vessel containing monomer liquid.

7. The device according to claim 1, wherein the operating element is operatively connected or connectable to the piston.

8. The device according to claim 1, wherein the operating element comprises a rotary joint.

9. The device according to claim 1, wherein the device is designed and configured to form a fluid-conducting connection between the base part and the cartridge by piercing the septum with the hollow mandrel in order to transfer a monomer liquid from the collection region to the cartridge.

10. The device according to claim 1, wherein the cartridge is detachably connectable to the base part.

11. The device according to claim 1, wherein the piston is movable by inserting the cartridge into the base part.

12. The device according to claim 1, wherein the base part comprises a stop for limiting the movement of the piston in the base part, wherein the operating element is preferably designed to engage in the stop.

13. The device according to claim 1, wherein the base part comprises a connecting channel with an exit opening, wherein the connecting channel forms a fluid-conducting connection between the container and the collection region.

14. The device according to claim 13, wherein the piston is designed and configured to close the exit opening of the connecting channel in a liquid-tight manner when the piston is inserted into the base part.

15. The device according to claim 1, wherein the device is designed and configured to transfer a monomer liquid from the base part to the cartridge while the base part is placed on a support such that the bottom part is oriented toward the support.
